(19) United States
(12) Patent Application Publication
(10) Pub. No.: US 2006/0046263 A1
(43) Pub. Date: Mar. 2, 2006

(54) Analysis

(75) Inventors: Peter David Gill, Birmingham (GB); Lindsey Ann Dixon, Birmingham (GB); Pieris Koumi, Birmingham (GB)

Correspondence Address: MERCHANT & GOULD PC, P.O. BOX 2903, MINNEAPOLIS, MN, 55402-0903, US

(73) Assignee: The Secretary of State for the Home Department, Birmingham (GB)

(21) Appl. No.: 11/212,370

(22) Filed: Aug. 25, 2005

(30) Foreign Application Priority Data
Sep. 2, 2004 (GB) 0419482.5

(51) Int. Cl. C12Q 1/68; G06F 19/00

(52) U.S. Cl. 435/6; 702/20

(57) ABSTRACT

A method for processing results, particularly from, the method comprising: obtaining from the results information concerning the single nucleotide polymorphisms implied for one or more loci, the information including identity information on the single nucleotide polymorphism or polymorphisms of a locus and a value related to the level detected for each identity; comparing each value with a first threshold and a second threshold, the comparison for the value or values for a locus determining the single nucleotide polymorphism identities considered to be possible for that locus.

This invention is concerned with improvements in and relating to analysis, particularly, but not exclusively the analysis of DNA using single nucleotide polymorphisms, SNP's.

According to a first aspect of the invention we provide a method for processing results, the method comprising:

obtaining from the results information concerning the single nucleotide polymorphisms implied for one or more loci, the information including identity information on the single nucleotide polymorphism or polymorphisms of a locus and a value related to the level detected for each identity;

comparing each value with a first threshold and a second threshold, the comparison for the value or values for a locus determining the single nucleotide polymorphism identities considered to be possible for that locus.

The method may include the collection and/or purification and/or amplification and/or analysis of a sample to provide the results. The method may be applied to results provided by others or previously obtained.

The information concerning the single nucleotide polymorphisms implied for one or more loci may imply the presence of two different single nucleotide polymorphism identities and/or the presence of one single nucleotide polymorphism identity and/or the presence of no single nucleotide polymorphism identities.

The single nucleotide polymorphism identities considered to be possible for that locus after the comparison may be the same as and/or different to and/or include additional identities when compared with the implied identities.

The identity information preferably indicates the single nucleotide polymorphism identity in terms of the implied presence of one or both bases forming the single nucleotide polymorphism.

The single nucleotide polymorphism identities considered to be possible for that locus after the comparison preferably indicates the single nucleotide polymorphism identity in terms of the one or both bases forming the single nucleotide polymorphism.

The value related to the level may be the peak height and/or the peak area for that identity.

Preferably the first threshold is higher than the second threshold.

The comparison may determine whether the value for an identity is greater than the first threshold and/or less than the first threshold and greater than the second threshold and/or less than the second threshold. Values equal to a threshold may be considered greater than the threshold. Values equal to a threshold may be considered less than the threshold.

The comparison may result in one from amongst one or more, preferably from amongst all of, the following determinations:—

a) p>A and q<B;

b) q>A and p<B;

c) p>A and q>B;

d) q>A and p>B;

e) p<A and p>B and q>B;

f) q<A and q>B and p>B;

g) p<A and p>B and q<B

h) q<A and q>B and p<B

I) p<B and q<B

where q is the value for one identity, p is the value for the other identity, A is the first, higher threshold and B is the second lower threshold.

The comparison may result in one from amongst one or more, preferably from amongst all of, the following determinations:—

a) p>A and q<B, the locus is homozygous for allele p;

b) q>A and p<B the locus is homozygous for allele q;

c) p>A and q>B the locus is heterozygous;

d) q>A and p>B the locus is heterozygous;

e) p<A and p>B and q>B the locus is heterozygous;

f) q<A and q>B and p>B the locus is heterozygous;

g) p<A and p>B and q<B the locus is homozygous for allele p or is heterozygous and allele q has dropped out;

h) q<A and q>B and p<B the locus is homozygous for allele q or is heterozygous and allele p has dropped out;

I) p<B and q<B the complete locus has dropped out and/or no statistically significant determination can be made;

where q is the value for one identity, p is the value for the other identity, A is the first, higher threshold and B is the second lower threshold.

The comparison may result in one from amongst one or more, preferably from amongst all of, the following determinations:—

a) p>A and q<B, then f=P*P;

b) q>A and p<B, then f=Q*Q;

c) p>A and q>B, then f=2*P*Q;

d) q>A and p>B, then f=2*P*Q;

e) p<A and p>B and q>B, then f=2*P*Q;

f) q<A and q>B and p>B, then f=2*P*Q;

g) p<A and p>B and q<B, then f=(P*P)+(2*P*Q);

h) q<A and q>B and p<B, then f=(Q*Q)+(2*P*Q);

I) p<B and q<B, then f=1;

where q is the value for one identity, p is the value for the other identity, A is the first, higher threshold and B is the second lower threshold, f is the match probability for that locus, P is the frequency of that identity in the population and/or a subset thereof, fro instance a database, Q is the frequency of that identity in the population and/or a subset thereof, fro instance a database. Such a comparison may provide a determination of the overall match probability between the sample that is the source of the results and a random source and/or another sample, potentially one whose source is known.

The comparison from one loci is preferably combined with the comparison from one or more other loci. The comparisons may be combined by multiplying a quantity obtained from the determination for each loci, for instance the match probability.

The comparison may be used to make a determination which establishes the genotype for the result and/or which quantifies the match probability for that result and/or which quantifies the extent of a match with another result and/or genotype and/or sample.

Preferably the method is applied to a plurality of different loci. The number of loci used may be at least 10, preferably is at least 15 and ideally is 20 or more. The loci may be analysed using a multiplex.

Preferably at least one of the thresholds has a value which is independent between loci. Preferably the first threshold value is independent between loci. Preferably the first threshold value for one locus is different from the first threshold value for one or more other loci.

Preferably the threshold value for a locus, and ideally at least the first threshold value therefore, is predetermined. Preferably the determination is provided according to the second aspect of the invention.

The first and/or second thresholds for the same locus may have different values for different method which are used to obtain the results, for instance due to different multi mixes being used between methods. The first and/or second thresholds for the same locus may have different values for different runs of the same method which are used to obtain the results, for instance due to a different batch of a multimix being used in one run compared with another.

According to a second aspect of the invention we provide a method for determining a threshold, the method comprising:

performing a plurality of analyses of the single nucleotide polymorphisms of a locus, the plurality of analyses including one or more analyses at a first feed sample quantity and one or more analyses at a second feed sample quantity;

determining a value related to the level of each single nucleotide polymorphism identity or identities detected for the first and second feed sample quantities;

selecting one of the values and determining the threshold from that value.

The threshold may be a threshold against which a comparison is made, preferably according to the first aspect of the invention. It is particularly preferred that the first threshold be determined in this way.

The first and/or second feed sample quantities may reflect the range of quantities preferred for analysis and possible for analysis. One of the feed sample quantities may be >500 pg/μL. One of the feed sample quantities may be 250 pg/μL. One of the feed sample quantities may be 125 pg/μL. One of the feed sample quantities may be <125 pg/μL. The feed sample quantities used may be these levels ±25%, or ±10%.

The value related to the level of each single nucleotide polymorphism identity or identities detected for the first and second feed sample quantities may be the peak height and/or peak area.

Preferably the value selected is one for which only one allele out of the two possible identities is observed. Preferably the value selected is one for which allele drop out is observed. Preferably the value selected is the highest value.

If both alleles are observed for all the fee sample quantities and/or allele drop out is not observed for any of the feed sample quantities then a further method may be used to determine the threshold. The further method may involve the determination of the heterozygous balance for that locus. The heterozygous balance may be established by taking the ratio of the lower value identity to the higher value identity under one or more conditions. The one or more conditions may be different feed sample quantities. The heterozygous balance for the locus may be used to predict the theoretical drop-out level for the locus. The value arising at the theoretical drop out level may be used as the selected value.

Preferably the threshold is determined from the selected value by applying a function to that value. The function may be a multiplier, for instance 1.2.

The method may further include performing a plurality of analyses of the single nucleotide polymorphisms of a locus, the plurality of analyses including one or more analyses with a first value for a further variable and one or more analyses with a second value for the further variable. The further variable may be injection time.

Preferably the method is used to determine the first and/or second thresholds for the same locus each time there is a change in the method which is used to obtain the results, for instance due to different multi mixes being used between methods. Preferably the method is used to determine the first and/or second thresholds for the same locus each time there is a change in a part of the method and/or component used therein and/or between different runs of the same method which are used to obtain the results, for instance due to a different batch of a multimix being used in one run compared with another.

Various embodiments of the invention will now be described, by way of example only, and with reference to the accompanying drawings in which:—

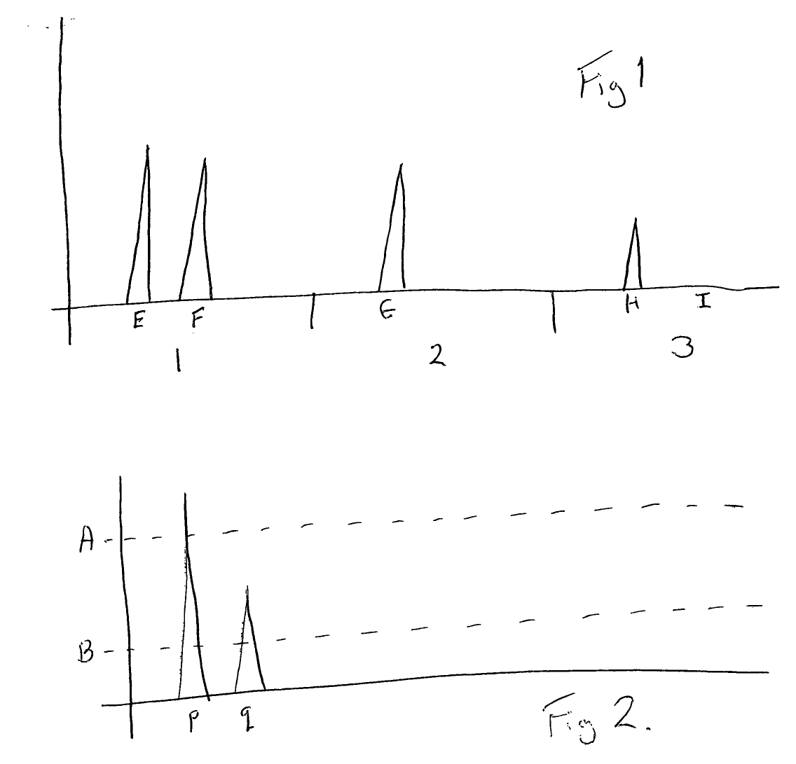

FIG. 1 is an illustration of allele result variation between loci;

FIG. 2 illustrates the thresholds used in interpreting the results according to an embodiment of the invention;

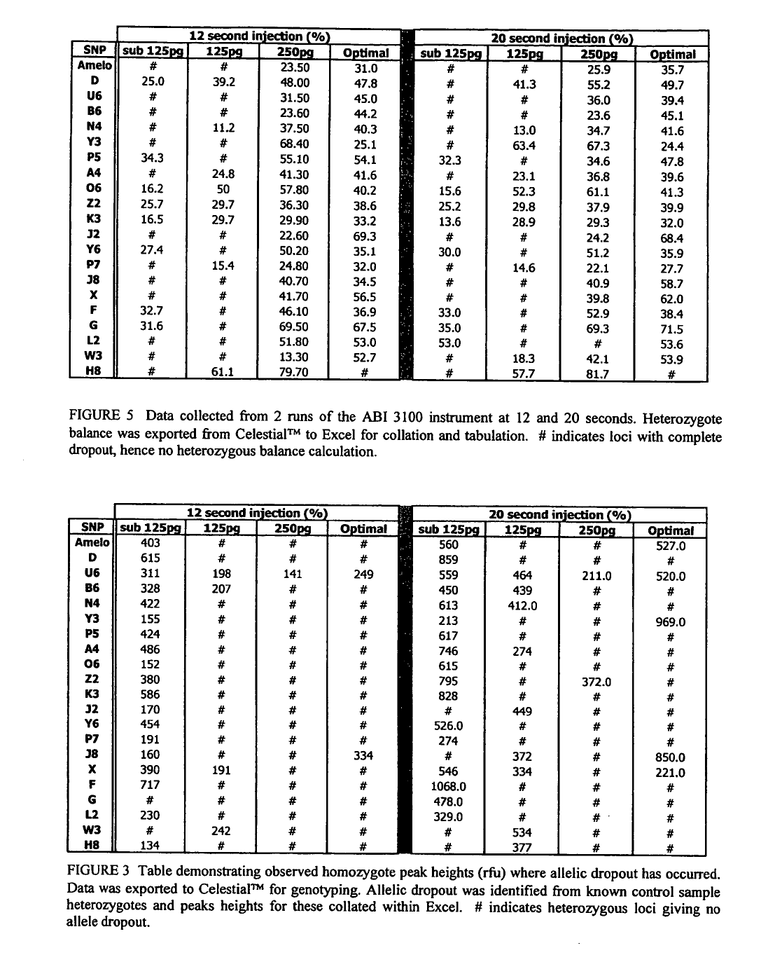

FIG. 3 illustrates variation in peak height with injection time and sample quantity for various loci when allele dropout occurs;

FIG. 4 illustrates heterozygous balance data and threshold data for use in implementing an embodiment of the invention, for various loci; and

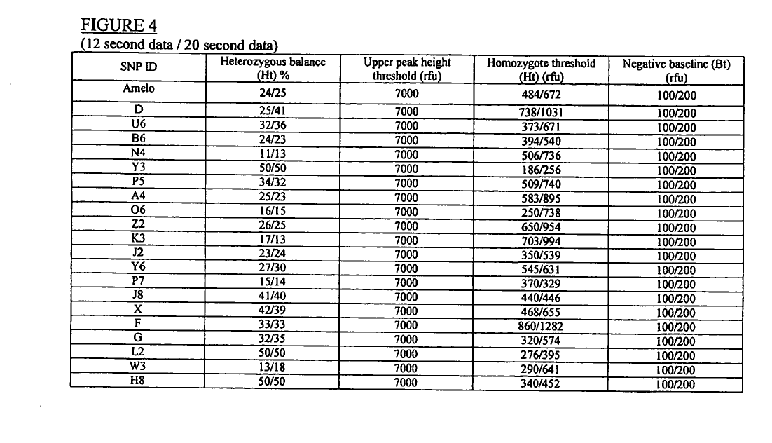

FIG. 5 illustrates heterozygous balance investigations with varying injection time and sample quantity for various loci.

The consideration of the identity present at a single nucleotide polymorphism site is useful for a variety of purposes, including medical diagnostics and forensic investigations. A sample to be analysed is amplified, marked in some way and then visualised to reveal the SNP identity at a particular locus. SNP consideration is particularly useful where STR (short tandem repeat) based analysis has not revealed a useful result, for instance due to the age of the sample.

Multiplexes are highly desirable to enable a large number of loci to be considered at the same time. Techniques for determining the identity of SNP's through the use of a multiplex are set out in WO01/07640, and specific primers for use in such a technique are set out in WO03/18831, the contents of both applications are incorporated herein by reference, particularly as they relate to the identity determining technique.

In situations where the sample being analysed contains low amounts of DNA and/or the DNA is degraded, then the results of the analysis process may indicate the identities of the SNP's in a way which requires interpretation.

In the illustrated example of FIG. 1, a sample from a single person is considered in respect of three loci. At a first locus 1, allele identity E and allele identity F are revealed so suggesting that the person is heterozygous for that locus. At a second locus 2, only allele G is revealed. Where this occurs, as it does in this case, with a strong indication for allele G, then that can reasonably be taken as indicated that the person is homozygous with respect to that locus. However, as indicated for the third locus 3, if the indication of a single identity H is weaker then there is uncertainty as to whether this is due to the person being homozygous for that locus or whether the person is heterozygous, but the other allele identity “I” is simply not detected.

To enable processing of such results by an expert system and/or to enable processing of such results without raising issues of subjectivity in the analysis, the present invention proposes a rule based approach to the interpretation.

The basic generic approach taken is illustrated with reference to FIG. 2. Here, with respect to a particular locus, the identities p and q are revealed by their respective peaks produced by the analysis and these are plotted on the X axis to reflect their identity. Their random fluoresence unit, rfu, value is plotted on the Y axis. The rfu values for each identity are compared with a first threshold, A, and a second lower threshold, B. The manner in which the thresholds are determined is described further below. Their comparison with the thresholds for the purpose of the interpretation is as follows:—

| If p>A and q<B then f=P*P; | (allele p exceeds A and locus |
|---|---|
| | is homozygous) |
| else if q>A and p<B then | (allele q exceeds B and locus |
| f=Q*Q; | is homozygous) |

| | |
|---|---|
| else if p>A and q>B then | (both alleles exceed B; locus |
| f=2*P*Q; | is heterozygous) |
| else if q>A and p>B then | (both alleles exceed B; locus |
| f=2*P*Q; | is heterozygous) |

| | |
|---|---|
| else if p<A and p>B and q>B | (both alleles exceed B; locus is |
| then f=2*P*Q; | heterozygous) |
| else if q<A and q>B and p>B | (both alleles exceed B; locus is |
| then f=2*P*Q; | heterozygous) |

| else if p<A and p>B and q<B | (allele q may |
|---|---|
| then f=(P*P)+(2*P*Q); | have dropped |
| | out) |
| else if q<A and q>B and p<B | (allele p may |
| then f=(Q*Q)+(2*P*Q); | have dropped |
| | out) |

| | |
|---|---|
| else if p<B and q<B then f=1; | (complete locus drop-out) |

where Q=1−P; f is the match probability, P is the population database frequency for that identity and Q is the population database frequency for that identity.

To get the results for a multiplex and/or for multiple loci, the f's for all the loci are multiplied together.

The population database frequencies are obtained by analysing a large number of samples so as to establish the frequency with which particular identities are observed.

As well as the beneficial rules provided above for use in interpretation, the present invention also provides for one or both of the threshold values being tailored between loci and/or when used in conjunction with different multimixes and/or even between different batches of the same multimix.

The manner in which this variation is provided for is now explained. Firstly, a series of analyses are run in which different known amounts of a sample are analysed. The amounts decrease from the “optimal level” normally used in SNP analysis to a fairly low level. Thus runs at 500 pg/μL, 250 pg/μL, 125 pg/μL and <125 pg/μL were performed. Two different sets of injection times were also used, 12 seconds and 20 seconds. The results are tabulated in FIG. 3. Where both alleles report, then the symbol # is used to denote no drop out in that run.

The maximum peak height occurring, preferably for one of the sub-500 pg/μL runs, for a run in which allele drop out occurs is of key interest. This value is taken and has 20% added to it to give the upper threshold A, for that locus at that injection time.

Thus, referring to FIG. 3, the maximum peak height was observed for U6, with a 12-second injection time, at sub 125 pg/μL and with a peak area of 311 rfu and with a 20 second injection time, at sub 125 pg/μL also, but with a peak height of 559 rfu. When these are each multiplied by 1.2 they give values of 373 and 671 respectively. These upper threshold values are tabulated in FIG. 4, together with other values for the upper thresholds for other loci obtained in the same way.

In some cases, drop-out was not observed for a locus at the optimal DNA template levels or sub-optimal levels. Locus G is an example of this. In such a case, the threshold value is obtained by using the heterozygous balance observed for that locus to predict the theoretical drop-out level for the locus. The peak height for that point, 266 in the case of locus G, again has 20% added to it to give the upper threshold, 320 for locus G.

The heterozygous balance is obtained by establishing the ratio of the smaller peak to the larger peak across a range of different amounts of sample and for different injection times. Thus 0 pg, 15.625 pg, 31.25 pg, 62.5 pg, 125 pg, 250 pg, 500 pg, 1 ng of sample were used in such tests. Typical results are set out in FIG. 5 and typical values are included in FIG. 4. Upper threshold values obtained in this way are also presented in FIG. 4.

The approach taken can be extended to the lower threshold, B, if desired.

A key benefit of the present invention is that it simplifies the design and operation of multiplexes. The design of multiplexes is already a difficult task due to requirements to balance amplification efficiencies, interactions between primers etc. Because the present invention enables the thresholds and/or interpretation is be variable between loci, this removes what would otherwise be a further constraint on multiplex design.

1. A method for processing results, the method comprising:
obtaining from the results information concerning the single nucleotide polymorphisms implied for one or more loci, the information including identity information on the single nucleotide polymorphism or polymorphisms of a locus and a value related to the level detected for each identity;
comparing each value with a first threshold and a second threshold, the comparison for the value or values for a locus determining the single nucleotide polymorphism identities considered to be possible for that locus.

2. A method according to claim 1 in which the value related to the level is the peak height or the peak area for that identity.

3. A method according to claim 1 in which the first threshold is higher than the second threshold.

4. A method according to claim 1 in which the comparison determines whether the value for an identity is greater than the first threshold and/or less than the first threshold ad greater than the second threshold and/or less than the second threshold.

5. A method according to claim 1 in which the comparison results in one from amongst one or more of the following determinations:—
a) p>A and q<B;
b) q>A and p<B;
c) p>A and q>B;
d) q>A and p>B;
e) p<A and p>B and q>B;
f) q<A and q>B and p>B;
g) p<A and p>B and q<B
h) q<A and q>B and p<B
I) p<B and q<B
where q is the value for one identity, p is the value for the other identity, A is the first, higher threshold and B is the second lower threshold.

6. A method according to claim 1 in which the comparison results in one from amongst one or more of the following determinations:—
a) p>A and q<B, the locus is homozygous for allele p;
b) q>A and p<B the locus is homozygous for allele q;
c) p>A and q>B the locus is heterozygous;
d) q>A and p>B the locus is heterozygous;
e) p<A and p>B and q>B the locus is heterozygous;
f) q<A and q>B and p>B the locus is heterozygous;
g) p<A and p>B and q<B the locus is homozygous for allele p or is heterozygous and allele q has dropped out;
h) q<A and q>B and p<B the locus is homozygous for allele q or is heterozygous and allele p has dropped out;
I) p<B and q<B the complete locus has dropped out and/or no statistically significant determination can be made;
where q is the value for one identity, p is the value for the other identity, A is the first, higher threshold and B is the second lower threshold.

7. A method according to claim 1 in which the comparison results in one from amongst one or more of the following determinations:—
a) p>A and q<B, then f=P*P;
b) q>A and p<B, then f=Q*Q;
c) p>A and q>B, then f=2*P*Q;
d) q>A and p>B, then f=2*P*Q;
e) p<A and p>B and q>B, then f=2*P*Q;
f) q<A and q>B and p>B, then f=2*P*Q;
g) p<A and p>B and q<B, then f=(P*P)+(2*P*Q);
h) q<A and q>B and p<B, then f=(Q*Q)+(2*P*Q);
I) p<B and q<B, then f=1;
where q is the value for one identity, p is the value for the other identity, A is the first, higher threshold and B is the second lower threshold, f is the match probability for that locus, P is the frequency of that identity in the population and/or a subset thereof, Q is the frequency of that identity in the population and/or a subset thereof.

8. A method according claim 1 in which the comparison provides a determination of the overall match probability between the sample that is the source of the results and a random source and/or another sample.

9. A method according to claim 1 in which at least one of the thresholds has a value which is independent between loci.

10. A method for determining a threshold, the method comprising:
performing a plurality of analyses of the single nucleotide polymorphisms of a locus, the plurality of analyses including one or more analyses at a first feed sample quantity and one or more analyses at a second feed sample quantity; determining a value related to the level of each single nucleotide polymorphism identity or identities detected for the first and second feed sample quantities; selecting one of the values and determining the threshold from that value.

11. A method according to claim 10 in which the threshold is a threshold against which a comparison is made comparing each value with a first threshold and a second threshold, the comparison for the value or values for a locus determining the single nucleotide polymorphism identities considered to be possible for that locus.

12. A method according to claim 10 in which one of the feed sample quantities is >500 pg/μL, one of the feed sample quantities is <500 pg/μL >250 pg/μL, one of the feed sample quantities is <250 pg/μL>125 pg/μL and one of the feed sample quantities is <125 pg/μL.

13. A method according to claim 10 in which if both alleles are observed for all the fee sample quantities and/or allele drop out is not observed for any of the feed sample quantities then a further method is used to determine the threshold.

14. A method according to claim 13 in which the further method involves the determination of the heterozygous balance for that locus, the heterozygous balance being established by taking the ratio of the lower value identity to the higher value identity under one or more conditions.

15. A method according to claim 14 in which the heterozygous balance for the locus is used to predict the theoretical drop-out level for the locus.
